(19) United States
(12) Patent Application Publication
(10) Pub. No.: US 2010/0226998 A1
(43) Pub. Date: Sep. 9, 2010

(54) ANTI-VIRAL AND ANTI-MICROBIAL DENTAL OPERATIVE MATERIAL AND METHODS

(75) Inventor: Dan E. Fischer, Sandy, UT (US)

Correspondence Address: Workman Nydegger;1000 Eagle Gate Tower, 60 East South Temple, Salt Lake City, UT, 84111, US

(73) Assignee: ULTRADENT PRODUCTS, INC., South Jordan, UT (US)

(21) Appl. No.: 12/398,227

(22) Filed: Mar. 5, 2009

(51) Int. Cl. A61K 33/40

(52) U.S. Cl. 424/616

(57) ABSTRACT

An anti-microbial and/or anti-viral composition for use in disinfecting an oral tissue during a dental procedure includes at least one dental operative material that is allowed to contact the oral tissue for a period of time between about 2 seconds and about 1 month and an effective amount of an anti-viral and/or anti-bacterial agent included in or mixed with the dental operative material so as to impart an anti-bacterial and/or anti-viral effect at or near a site of administration in oral tissue. The anti-viral and/or anti-bacterial agent at least partially at least partially contributes to a two-log (or three-log, four-log, or five-log) reduction in viral and/or bacterial activity at or near a site of administration. The compositions protect a dental practitioner and/or other patient from being infected by a communicable disease carried by a treated patient.

### BACKGROUND OF THE INVENTION

1. The Field of the Invention

The present invention relates generally to devices and methods for preventing infection following a dental procedure. In particular, the invention relates to anti-viral and/or anti-bacterial dental operative materials that can be applied to oral tissues during a dental procedure. Anti-viral and/or anti-bacterial components in the dental operative materials can also diffuse into the mouth to reduce the numbers of viable viruses and bacteria in the mouth.

2. The Relevant Technology

The mouth has a rich blood supply and the oral tissues are kept constantly moist by saliva. The rich blood supply and the constant moistening by saliva are necessary for overall oral health, but, as a result, the mouth provides a favorable breeding ground for pathogens such as viruses and bacteria. Given the right circumstances, these pathogens can cause infection of exposed tissues in the mouth and transmit infection to other people.

Most oral tissues are relatively tender and dental procedures commonly cause some degree of injury and bleeding to sensitive oral tissues found in the mouth. Oral tissues injured in dental procedures can become infected by virus- and bacteria-laden saliva if proper precautions are not taken.

For example, the gums can often be nicked and bloodied during dental procedures such as teeth cleaning or filling a cavity. Such wounds can become infected in some cases, causing harm to the patient. Moreover, blood from nicks or cuts can infect others, such as the dental practitioner and/or be transferred to other patients.

In another example, dentists often need to take accurate impression of the teeth in preparation for making prosthetics. In order to take an accurate impression, however, it is often necessary to retract the gums from the teeth surround the area where the prosthetic will be inserted. In a typical retraction, the gums are separated from the teeth and retracted by packing them with a cord material. This retraction inevitably injures the gums and can cause bleeding. As with the above example, such injured areas can become infected in some cases and the blood that from the injured areas can act as an infectious agent.

In yet another example, when tooth decay or a cracked tooth results in an infected tooth, the pulp (the soft structure of a tooth, consisting of nerves, blood vessels, and connective tissue) generally must be removed using an endodontic procedure (i.e., a root canal) in order to preserve the tooth. An opening is made through the crown of the tooth into the pulp canal and as much of the pulp material as is possible is removed from the pulp canal of the tooth. The presence of bacteria is minimized through the use of irrigants and dressings to avoid infection. These steps are all done to form and prepare the pulp cavity for sealing or obturation, which involves filling the pulp cavity with biocompatible materials, such as gutta percha, before the pulp cavity is sealed.

It is often necessary, however, to apply a temporary filling and seal over a cleansed and hollowed pulp canal so that a patient can be given medicine to eliminate infections that may have spread to deep portions of the pulp canal or beyond the tooth. The use of the temporary restoration allows a dental practitioner to ensure that the infection is eliminated before the final restoration is applied. However, such temporary restorations can leak allowing bacteria-laden saliva to enter the pulp canal and re-infect the tooth.

Saliva and blood can also act as infectious agents that can infect practitioners as well as other patients. This is especially true for patients that may be infected with viral illnesses such as hepatitis, herpes, and/or HIV/AIDS. Practitioners need to be confident that they will not be infected in the course of treating their patients and that they will not infect other patients (i.e., through cross contamination). Moreover, immune compromised patients such as those suffering from HIV/AIDS are particularly susceptible to infection, and any exposed tissue in the mouth can be infected by exposure to the saliva. That is, they are particularly susceptible to infection and their blood and saliva can be particularly infectious. Any one or a combination of the dental operative materials discussed above can be applied to oral tissues being treated in order to stem the spread of infectious agents.

### BRIEF SUMMARY OF THE INVENTION

Saliva and other oral fluids are often laden with viruses and bacteria and, as such, exposed oral tissues can become infected during dental procedures. Likewise, bacteria- and virus-laden saliva, blood, and other oral fluids can transmit infection from patient-to-doctor and sometimes even patient-to-patient through cross contamination. The present invention provides anti-viral and anti-bacterial dental operative materials for preventing the spread of infectious agents during or after a dental procedure and related methods for preventing the spread of infectious agents.

One embodiment of the present invention includes an anti-viral and/or anti-bacterial device or composition for use in disinfecting oral tissue during a dental procedure. In one embodiment, the anti-viral and/or anti-bacterial device or composition can include at least one dental operative material. The dental operative material is characterized by relatively short-term contact with oral tissue during a dental procedure (i.e., from as little as two seconds to as long as about one month, most typically about five seconds to about two minutes), with an effective amount of an anti-viral and/or anti-bacterial agent included in the dental operative material so as to impart an anti-bacterial and/or anti-viral effect at a site of administration.

Dental operative materials that can be included in the claimed device include substances that can be placed into the mouth during a dental procedure that are allowed to temporarily contact oral tissues, blood, and other oral fluids. A typical dental operative material may be allowed to contact an oral tissue for a period of time ranging from a few seconds to a few days. Because they contact exposed oral tissues during dental procedures, dental operative materials can be effectively used to deliver anti-viral and/or anti-microbial materials to exposed oral tissues. Moreover, because dental operative materials only remain in the patient's mouth for relatively short time periods, there is little long-term risk of poisoning or toxicity by the anti-bacterial and/or anti-viral agent.

Examples of suitable dental operative materials include, but are not limited to, dental stains, caries detecting stains, dental disinfectant solutions, dental disinfectant gels, dental disinfectant slurries, dental disinfectant pastes, dental hemostatic solutions, root canal disinfectants, root canal chelation solutions, dental polishes, dental bonding primers, dental lubricants, dental lubricant/chelation solutions, oxygen inhibiting dental primers, polymerizable dental materials, dental caulking materials, dental putties, acidic dental etching solutions, dental cements, root canal obturation agents, dental prosthetic bonding agents, and combinations thereof. Disinfectant compositions according to the invention are characterized by their ability to prevent the spread of communicable diseases (e.g., systemic diseases, such as AIDS, herpes and the like), such as from the patient to the doctor and/or from patient to patient through cross contamination, not merely killing bacteria normally found in a healthy patient's mouth as with typical dental disinfectant compositions.

Examples of suitable anti-viral and/or anti-bacterial compounds that can be included in the dental operative material include, but are not limited to, chlorhexadine, benzalkonium chloride, benzethonium chloride, quaternary ammonium compounds, quaternary ammonium halide compounds, sodium hypochlorite, 1,1′-hexamethylene bis(5(p-chlorophenyl)biguanide), cetyl pyridinium chloride, cetyl pyridinium bromide, methyl 4-hydroxybenzoate, propylparaben (propyl p-hydroxybenzoate), hydrogen peroxide, phenol, and combinations thereof. In a preferred embodiment, the anti-viral and/or the anti-bacterial compound is benzalkonium chloride, benzethonium chloride, another quaternary ammonium compound, or another quaternary ammonium halide compound.

In one embodiment, the anti-viral and/or the anti-bacterial compound has a concentration in a range of about 0.1% to about 10% by weight in the dental operative material, preferably a concentration in a range of about 0.2% to about 6% by weight in the dental operative material, and more preferably a concentration in a range of about 1% to about 3% by weight in the dental operative material.

In one embodiment, the present invention includes an anti-viral and/or anti-bacterial device for use in disinfecting an oral tissue during and/or following a dental procedure so as to produce at least a two-log reduction in viral and/or bacterial activity at or near a site of administration, preferably at least a three-log reduction, more preferably at least a four-log reduction, and most preferably at least a five-log reduction in viral and/or bacterial activity at or near the site of administration.

In one embodiment, a method for preventing infection during and/or following a dental procedure includes (1) exposing at least one oral tissue, and (2) contacting the exposed oral tissue with at least one anti-viral and/or anti-bacterial dental operative material, the dental operative material being characterized by short-term contact with an oral tissue during a dental procedure, and an effective amount of an anti-viral and/or anti-bacterial agent so as to impart an anti-bacterial and/or anti-viral protective effect at a site of administration in oral tissue. The effective amount of the anti-viral and/or anti-bacterial agent provides at least a two-log reduction in viral and/or bacterial activity at or near the site of administration, preferably at least a three-log reduction, more preferably at least a four-log reduction, and most preferably at least a five-log reduction in viral and/or bacterial activity at or near the site of administration.

In one embodiment, the dental operative material is allowed to contact the oral tissue for a period of time between about 2 seconds to about 1 month (e.g., up to about two weeks in the case of a temporary dental material placed in a root canal of a tooth prior to installing the permanent crown), preferably about 3 seconds to about 5 days, more preferably about 4 seconds to about 1 hour, and most preferably about 5 seconds to about 2 minutes.

These and other advantages and features of the present invention will become more fully apparent from the following description and appended claims, or may be learned by the practice of the invention as set forth hereinafter.

### DETAILED DESCRIPTION OF THE PREFERRED EMBODIMENTS

### I. Introduction and Definitions

Saliva and other oral fluids are often laden with viruses and bacteria and, as such, exposed oral tissues can become infected during dental procedures. Likewise, bacteria- and virus-laden saliva, blood, and other oral fluids can transmit infection from patient-to-doctor and/or patient-to-patient through cross contamination. The present invention provides dental operative materials for preventing spread of infectious agents during or following a dental procedure. Methods for preventing spread of infectious agents during or after a dental procedure using an anti-viral or anti-bacterial dental operative material are also provided.

The term “dental operative material” is used herein to describe substances that are typically placed into the mouth during a dental procedure that may be allowed to contact an oral tissue, blood, and/or other oral fluids for a period of time ranging from a few seconds to a few weeks. A dental operative material is generally not designed to remain permanently in a patient's mouth (e.g., not longer than about one month).

II. Anti-Viral and/or Anti-Bacterial Dental Operative Materials

One embodiment of the present invention includes an anti-microbial device for use in disinfecting an oral tissue during a dental procedure. In one embodiment, the anti-microbial device can include at least one dental operative material and an effective amount of an anti-viral and/or anti-bacterial agent included in the dental operative material so as to impart an anti-bacterial and/or anti-viral protective effect at or near a site of administration in oral tissue.

In one embodiment, the effective amount of the anti-viral and/or anti-bacterial agent is sufficient to produce at least a two-log reduction in viral and/or bacterial activity at or near the site of administration, preferably at least a three-log reduction, more preferably at least a four-log reduction, and most preferably at least a five-log reduction in viral and/or bacterial activity at or near the site of administration. A two-log reduction represents a 100-fold (i.e., 10²) or 99% drop in the number of viable viruses and/or bacteria at or near the site of administration of the dental operative material. A three-log reduction represents a 1000-fold (i.e., 10³) or 99.9% drop in the number of viable viruses and/or bacteria at or near the site of administration of the dental operative material. A four-log reduction represents a 10,000-fold (i.e., 10⁴) or 99.99% drop in the number of viable viruses and/or bacteria at or near the site of administration of the dental operative material. A five-log reduction represents a 100,000-fold (i.e., 10⁵) or 99.999% drop in the number of viable viruses and/or bacteria at or near the site of administration of the dental operative material.

According to one embodiment, the reduction in viral and/or bacterial activity may be provided “step-wise”. For example, the dental operative material may itself be capable of killing some level of viruses and/or bacteria at or near the site of administration of the dental operative material. The anti-viral and/or anti-bacterial agent may then boost the kill rate to a higher level, such as to kill communicable or systemic diseases in addition to simply killing bacteria normally found in a healthy patient's mouth. Thus, a portion of the total kill rate may be provided by the inherent anti-viral and/or anti-bacterial properties of the dental operative material and another portion of the total kill rate is then provided by the additional anti-viral and/or anti-bacterial agent.

By way of example, the dental operative material may, by virtue of its raw chemistry, be capable of providing a 1.5 log reduction in viruses and/or bacteria at or near the site of administration of the dental operative material. To boost the kill rate and provide at least a two-log reduction in viruses and/or bacteria, the dental operative material must be supplemented by the addition of a supplemental anti-viral and/or anti-bacterial agent. Similarly, if the dental operative material inherently provides a 2.5-log reduction in viruses and/or bacteria at or near the site of administration of the dental operative material, but a three-log reduction or more in viruses and/or bacteria is desired, the inventive dental operative material must be supplemented by the addition of a supplemental anti-viral and/or anti-bacterial agent to provide the desired kill rate.

In general, the supplemental anti-viral and/or anti-bacterial agent required to provide a desired kill rate will generally be included in an amount so to boost the overall, or step-wise, kill rate by at least about a half-log reduction in viruses and/or bacteria at or near the site of administration of the dental operative material, preferably to achieve an overall kill rate of about a 1-5 log reduction, more preferably about a 2-4 log reduction, and most preferably about a 3-log reduction in viruses and/or bacteria at or near the site of administration of the dental operative material.

Examples of suitable anti-viral and/or the anti-bacterial compounds that can be included in the dental operative material include, but are not limited to, chlorhexadine, benzalkonium chloride, benzethonium chloride, quaternary ammonium compounds, quaternary ammonium halide compounds, sodium hypochlorite, 1,1′-hexamethylene bis(5(p-chlorophenyl)biguanide), cetyl pyridinium chloride, cetyl pyridinium bromide, methyl 4-hydroxybenzoate, propylparaben (propyl p-hydroxybenzoate), hydrogen peroxide, phenol, and combinations thereof. In a preferred embodiment, the anti-viral and/or the anti-bacterial compound is benzalkonium chloride, benzethonium chloride, or another quaternary ammonium compound.

Quaternary ammonium compounds are positively charged polyatomic ions of the structure NR₄⁺ with R₄ representing four alkyl groups that can be the same or different than one another. Unlike the ammonium ion (i.e., NH₄⁺) and primary, secondary, or tertiary ammonium cations, quaternary ammonium compounds are permanently charged, independent of the pH of their solution. Quaternary ammonium compounds are typically used as disinfectants, surfactants, fabric softeners, and as antistatic agents (e.g., in shampoo). Certain long alkyl chain quaternary ammonium compounds are used as antimicrobials and disinfectants. Examples include benzalkonium chloride, benzethonium chloride, methylbenzethonium chloride, cetalkonium chloride, cetylpyridinium chloride, cetrimonium, cetrimide, dofanium chloride, tetraethylammonium bromide, didecyldimethylammonium chloride, and domiphen bromide. Quaternary ammonium compounds act by disrupting the cell membranes and proteins of viruses and/or bacteria. Quaternary ammonium compounds are effective against virtually all viruses and bacteria except endospores, Mycobacteria (e.g., M. tuberculosis), lipid-containing viruses, and Pseudomonads.

Examples of suitable quaternary ammonium halide compounds include, but are not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, tetradecyltrimethylammonium halides, alkylbenzyldimethylammonium salts alkyltrimethylammonium bromide, benzyldimethylhexadecylammonium chloride, benzyldimethyltetradecylammonium chloride, benzyldodecyldimethylammonium bromide, benzyltrimethylammonium tetrachloroiodate, dimethyldioctadecylammonium bromide, dodecylethyldimethylammonium bromide, dodecyltrimethylammonium bromide, dodecyltrimethylammonium bromide, ethylhexadecyldimethylammonium bromide, hexadecyltrimethylammonium bromide, hexadecyltrimethylammonium bromide, trimethyl(tetradecyl)ammonium bromide, and combinations comprising one or more of the foregoing compounds. Examples of suitable halides include chloride, fluoride, bromide and iodide.

In one embodiment, the anti-viral and/or the anti-bacterial compound has a concentration in a range of about 0.1% to about 10% by weight in the dental operative material, or preferably a concentration in a range of about 0.2% to about 6% by weight in the dental operative material, or more preferably a concentration in a range of about 1% to about 3% by weight in the dental operative material.

In general the anti-viral and/or the anti-bacterial compounds discussed herein are capable of producing a two-log reduction in the numbers of viable viruses and/or bacteria at or near the site of administration within about 30 seconds or less (i.e., 99% of viable viruses or bacteria are killed within about 30 seconds or less). Preferably, the anti-viral and/or the anti-bacterial compounds discussed herein are capable of producing a three-log reduction in the numbers of viable viruses and/or bacteria at or near the site of administration within about 30 seconds or less (i.e., 99.9% of viable viruses or bacteria are killed within about 30 seconds or less). More preferably, the anti-viral and/or the anti-bacterial compounds discussed herein are capable of producing a four-log reduction in the numbers of viable viruses and/or bacteria at or near the site of administration within about 30 seconds or less (i.e., 99.99% of viable viruses or bacteria are killed within about 30 seconds or less). Most preferably, the anti-viral and/or the anti-bacterial compounds discussed herein are capable of producing a five-log reduction in the numbers of viable viruses and/or bacteria at or near the site of administration within about 30 seconds or less (i.e., 99.999% of viable viruses or bacteria are killed within about 30 seconds or less).

Many preparations contain preservative amounts (e.g., about 0.005 wt % to about 0.01 wt %) of anti-viral and/or anti-bacterial compounds. While such, these low concentrations may help prevent spoilage of the preparation, they are not sufficient to produce a two-log to five-log reduction in the numbers of viable bacteria when the preparation is applied to a surface (e.g., an exposed oral tissue). Other compositions may, in fact, be designed to kill bacteria normally found in a healthy patient's mouth and might in fact be capable of producing a two-log or more reduction in such bacteria. However, such compositions are generally not capable of treating communicable diseases found systemically in a sick person's blood. Such compositions therefore may not effectively prevent the spread of communicable diseases from patient-to-practitioner and/or from patient to patient via cross contamination. By including supplemental anti-viral and/or anti-bacterial agents, the present invention boosts the ability of such compositions to prevent the spread of communicable diseases rather than simply killing bacteria normally found in the mouth of otherwise healthy patients (i.e., to provide a superior environment to treat and promote healthy teeth).

Examples of suitable dental operative materials that can be used in the anti-viral and/or anti-bacterial device disclosed herein include, but are not limited to, dental stains, caries detecting stains, dental disinfectant solutions, dental disinfectant gels, dental disinfectant slurries, dental disinfectant pastes, dental hemostatic agents, root canal disinfectants, root canal chelation solutions, dental polishes, dental bonding primers, dental lubricants, dental lubricant/chelation solutions, oxygen inhibiting dental primers, impression materials, polymerizable dental materials, dental caulking materials, dental putties, acidic dental etching solutions, dental cements, root canal obturation agents, or dental prosthetic bonding agents, and combinations thereof.

Examples of suitable dental stain and caries detecting stain products include Seek™ and Sable Seek™ from Ultradent Products, Inc. Seek™ and Sable Seek™ are designed to stain demineralized dentin (i.e., dental caries) to allow easy removal of the decayed portions of the tooth. Soft tissues along the gum line and in the tooth are often exposed in the process of removing the decayed portions of the tooth, and Seek™ and Sable Seek™ are designed to help practitioners differentiate soft tissue from demineralized dentin.

Examples of suitable dental disinfectant solutions, dental disinfectant gels, dental disinfectant slurries, dental disinfectant pastes include, and root canal disinfectants include Consepsis™, Consepsis Scrub™ ChlorCid™, and UltraCal™ from Ultradent Products, Inc. Consepsis™ and Consepsis Scrub™ are general oral irrigation and cleansing products. ChlorCid™ and UltraCal™ are designed for irrigating and disinfecting a root canal prior to filling the tooth with an obturation cement. As discussed above, dental disinfectant compositions according to the invention are formulated so as to achieve the desired reduction in the number of viruses and/or bacteria by including supplemental anti-viral and/or anti-bacterial agents.

Examples of suitable dental hemostatic agents include Viscostat™ and Astringident™ ferric sulfate-based hemostatic agents available from Ultradent Products, Inc. Viscostat™ and Astringident™ are applied to tissues to control bleeding during dental procedures. Another suitable example of a dental hemostatic agent is Ultrapak™ cord, which is also available from Ultradent Products, Inc. Ultrapak™ cord is used for rapid tissue displacement prior to impression-making, and can also be used to deliver ferric sulfate solutions subgingivally, for sulcular fluid control.

Examples of suitable root canal chelation solutions, dental lubricants, dental lubricant/chelation solutions include EDTA solution, citric acid solution, and File-Eze™ available from Ultradent Products, Inc. EDTA and citric acid solutions are root canal chelating agents that condition/clean through a chelation process. File-Eze™ is an EDTA-containing solution for chelating root canals while providing lubrication to facilitate safely debriding the root canal.

Examples of suitable dental polishes include Ultradent Diamond Polish™, which is available from Ultradent Products, Inc.

Examples of suitable dental bonding primers include PrimaDry™, which is available from Ultradent Products, Inc.

Examples of suitable oxygen inhibiting dental primers include DeOx™, which is available from Ultradent Products, Inc.

Examples of suitable impression materials include PrepQuik™ and Chromaclone Alginate™, which are available from Ultradent Products, Inc. PrepQuik™ is a glycolic acid solution that can e applied to the teeth and gum area prior to taking an impression in order to improve the quality of the impression. Chromaclone Alginate™ is the material in which the impression is taken.

Examples of suitable polymerizable dental operative materials include OpalDam™, which is available from Ultradent Products, Inc. OpalDam™ is a light-cured methacrylate-based polymer that can be temporarily applied to the gums around the teeth in order to isolate and protect the gums during a dental procedure (e.g., tooth bleaching using caustic peroxide compositions).

Examples of suitable dental caulking materials and dental putties include OraSeal™ caulk and OraSeal™ putty, which are available from Ultradent Products, Inc. OraSeal™ caulk and OraSeal™ putty can be used similarly to OpalDam™.

Examples of suitable dental etching solutions include UltraEtch™, which is phosphoric acid etchant available from Ultradent Products, Inc. Because highly acidic compositions can themselves be anti-pathogenic, it would not be apparent to one of ordinary skill in the art why it might be desirable to add one or more of the anti-bacterial and/or anti-viral agents disclosed herein. The reason is to ensure at least a three-log or five-log decrease in the number of bacterial and/or viruses at the site of the dental procedure. Acids, when neutralized, can lose their inherent anti-pathogenic activity and may therefore not ensure the desired reduction in the number of viruses and/or bacteria at the dental procedure site.

Examples of suitable dental cements, root canal obtruation agents, and/or dental prosthetic bonding agents include UltraTemp™, Gutta Percha, and EndoRez™, which are available from Ultradent Products, Inc. UltraTemp™ is a temporary luting agent/filling material that can be used for temporary restoratives. Gutta Percha and EndoRez™ can be used as semi-permanent or permanent root canal filling materials. They are, however, sealed within the interior of a non-vital tooth, thereby shielding the patient from potentially toxic effects of the anti-viral and/or anti-bacterial agent.

A characteristic common to all of the dental operative materials listed above is that they are placed into a patient's mouth during a dental procedure where they can contact exposed oral tissues and any blood or other fluids that are produced in a dental procedure. The dental operative materials are typically allowed to contact oral tissues, blood, and/or other oral fluids for a period of time ranging from a few seconds to a few days. Because they contact oral tissues exposed during dental procedures, dental operative materials can be effectively used to deliver anti-viral and/or anti-bacterial materials to exposed oral tissues. Moreover, in some cases, the dental operative material can stay in contact with the exposed tissue until the tissue heals, providing further protection from infection. Because the anti-viral and/or anti-bacterial compounds in the dental operative materials can typically diffuse into the mouth, the dental operative materials can also be used to kill infectious agents carried in a patient's oral fluids or blood in order to prevent transmission of disease from patient-to-practitioner and patient-to-patient.

In one embodiment, the dental operative material is formulated so as to contact the oral tissue for a period of time between about 2 seconds and about 1 month. Preferably, the dental operative material is allowed to contact the oral tissue for a period of time between about 3 seconds to about 4 days, more preferably about 4 seconds to about one hour, and most preferably the dental operative material is allowed to contact the oral tissue for a period of time between about 5 seconds and about 2 minutes.

### III. Exemplary Methods of Use

In order to prevent infections in the mouth and in order to prevent spread of infectious agents from patient-to-patient and/or from patient-to-doctor, a method for preventing infection following a dental procedure is disclosed. The method includes (1) exposing at least one oral tissue, and (2) contacting the exposed oral tissue with at least one anti-viral and/or anti-bacterial material, the anti-viral and/or anti-bacterial material including: (a) at least one dental operative material, the dental operative material being characterized by short-term contact with an oral tissue during a dental procedure, and (b) an effective amount of an anti-viral and/or anti-bacterial agent included in the dental operative material so as to impart an anti-bacterial and/or anti-viral protective effect at a site of administration in oral tissue, the effective amount of the anti-viral and/or anti-bacterial agent producing at least a two-log reduction in viral and/or bacterial activity at or near the site of administration, preferably at least a three-log reduction, more preferably at least a four-log reduction, and most preferably at least a five-log reduction in viral and/or bacterial activity at or near the site of administration.

In one embodiment, the method further includes contacting the exposed oral tissue with the dental operative material for a period of time between about 2 seconds and about 1 month, preferably between about 3 seconds to about 4 days, more preferably between about 4 seconds to about one hour, and most preferably between about 5 seconds and about 2 minutes.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An anti-viral and/or anti-bacterial composition for use in disinfecting oral tissue during and/or after a dental procedure, the composition comprising:
at least one dental operative material, the dental operative material being designed for short-term contact with oral tissue during and/or after a dental procedure; and
an effective amount of an anti-viral and/or anti-bacterial agent included in or mixed with the dental operative material so as to at least partially contribute to the anti-viral and/or anti-bacterial composition imparting an anti-bacterial and/or anti-viral effect at a site of administration in oral tissue and producing at least a two-log reduction in viral and/or bacterial activity at the site of administration.

2. An antibacterial device as recited in claim 1, the effective amount of the anti-viral and/or anti-bacterial agent at least partially contributing to the anti-viral and/or anti-bacterial composition producing at least a three-log reduction in viral and/or bacterial activity at the site of administration.

3. An antibacterial device as recited in claim 1, the effective amount of the anti-viral and/or anti-bacterial agent at least partially contributing to the anti-viral and/or anti-bacterial composition producing at least a four-log reduction in viral and/or bacterial activity at the site of administration.

4. An antibacterial device as recited in claim 1, the effective amount of the anti-viral and/or anti-bacterial agent at least partially contributing to the anti-viral and/or anti-bacterial composition producing at least a five-log reduction in viral and/or bacterial activity at the site of administration.

5. An antibacterial device as recited in claim 1, wherein the anti-viral and/or the anti-bacterial agent is selected from the group consisting of chlorhexadine, benzalkonium chloride, benzethonium chloride, quaternary ammonium salts, quaternary ammonium halide salts, sodium hypochlorite, 1,1′-hexamethylene bis(5(p-chlorophenyl)biguanide), cetyl pyridinium chloride, cetyl pyridinium bromide, methyl 4-hydroxybenzoate, propylparaben (propyl p-hydroxybenzoate), hydrogen peroxide, phenol, and combinations thereof.

6. An antibacterial device as recited in claim 1, wherein the anti-viral and/or the anti-bacterial agent has a concentration in a range of about 0.1% to about 10% by weight in the dental operative material.

7. An antibacterial device as recited in claim 1, wherein the anti-viral and/or the anti-bacterial agent has a concentration in a range of about 0.2% to about 6% by weight in the dental operative material.

8. An antibacterial device as recited in claim 1, wherein the anti-viral and/or the anti-bacterial agent has a concentration in a range of about 1% to about 3% by weight in the dental operative material.

9. An antibacterial device as recited in claim 1, wherein the dental operative material is selected from the group consisting of a dental stain, a caries detecting stain, a dental disinfectant solution, a dental disinfectant gel, a dental disinfectant slurry, a dental disinfectant paste, a dental hemostatic solution, a root canal disinfectant, a root canal chelation solution, a dental polish, a dental bonding primer, a dental lubricant, a dental lubricant/chelation solution, an oxygen inhibiting dental primer, a polymerizable dental material, a dental caulking material, a dental putty, an acidic dental etching solution, a dental cement, a root canal obturation cement, a dental prosthetic bonding cement, and combinations thereof.

10. An antibacterial device as recited in claim 1, wherein the dental operative material is designed to contact oral tissue for a period of time between about 2 seconds and about one month.

11. An antibacterial device as recited in claim 1, wherein the dental operative material is designed to contact the oral tissue for a period of time between about 3 minute and about 5 days.

12. An antibacterial device as recited in claim 1, wherein the dental operative material is designed to only contact the oral tissue for a period of time between about 5 seconds and about 2 minutes.

13. An anti-viral and/or antibacterial composition for use in disinfecting oral tissue during and/or after a dental procedure, the composition comprising:
at least one dental operative material, the dental operative material being designed to only contact oral tissue for a period of time between about 2 seconds and about 30 days; and
an anti-viral and/or anti-bacterial agent included in or mixed with the dental operative material at a concentration in a range of about 0.1% to about 10% by weight so as to at least partially contribute to the anti-viral and/or antibacterial composition producing at least a three-log reduction in viral and/or bacterial activity at or near a site of administration.

14. An anti-viral and/or antibacterial composition as recited in claim 13, wherein the anti-viral and/or anti-bacterial composition produces at least a four-log reduction in viral and/or bacterial activity at or near the site of administration.

15. An anti-viral and/or antibacterial composition as recited in claim 13, wherein the anti-viral and/or the anti-bacterial agent is selected from the group consisting of chlorhexadine, benzalkonium chloride, benzethonium chloride, quaternary ammonium salts, quaternary ammonium halide salts, sodium hypochlorite, 1,1′-hexamethylene bis(5(p-chlorophenyl)biguanide), cetyl pyridinium chloride, cetyl pyridinium bromide, methyl 4-hydroxybenzoate, propylparaben (propyl p-hydroxybenzoate), hydrogen peroxide, phenol, and combinations thereof.

16. An anti-viral and/or antibacterial composition as recited in claim 13, wherein the dental operative material is selected from the group consisting of a dental stain, a caries detecting stain, a dental disinfectant solution, a dental disinfectant gel, a dental disinfectant slurry, a dental disinfectant paste, a dental hemostatic solution, a root canal disinfectant, a root canal chelation solution, a dental polish, a dental bonding primer, a dental lubricant, a dental lubricant/chelation solution, an oxygen inhibiting dental primer, a polymerizable dental material, a dental caulking material, a dental putty, an acidic dental etching solution, a dental cement, a root canal obturation cement, or a dental prosthetic bonding cement, and combinations thereof.

17. A method for preventing infection by a treated patient of a dental practition and/or other patent during or following a dental procedure, the method comprising:
exposing at least one oral tissue at an administration site of a treated patient; and
contacting the exposed oral tissue at the administration site with at least one anti-viral and/or anti-bacterial composition for a time period between about 2 seconds and about 1 month, the anti-viral and/or anti-bacterial composition including:
at least one dental operative material; and
an effective amount of an anti-viral and/or anti-bacterial agent included in or mixed with the dental operative material so as to at least partially contribute to the anti-viral and/or anti-bacterial composition providing an anti-bacterial and/or anti-viral effect at the administration site; and
causing or allowing the anti-viral and/or anti-bacterial composition to provide an anti-bacterial and/or anti-viral effect so as to produce at least a two-log reduction in viral and/or bacterial activity at or near the administration site.

18. A method as recited in claim 17, the anti-viral and/or anti-bacterial composition producing at least a three-log reduction in viral and/or bacterial activity at or near the administration site.

19. A method as recited in claim 17, the anti-viral and/or anti-bacterial composition producing at least a four-log reduction in viral and/or bacterial activity at or near the administration site.

20. A method as recited in claim 17, the anti-viral and/or anti-bacterial composition producing at least a five-log reduction in viral and/or bacterial activity at or near the administration site.

21. A method as recited in claim 17, the exposed oral tissue being contacted with the at least one anti-viral and/or anti-bacterial composition for a period of time between about 3 seconds and about 5 days.

22. A method as recited in claim 17, the exposed oral tissue being contacted with the at least one anti-viral and/or anti-bacterial composition for a period of time between about 4 seconds and about 1 hour.

23. A method as recited in claim 17, the exposed oral tissue being contacted with the at least one anti-viral and/or anti-bacterial composition for a period of time between about 5 seconds and about 2 minutes.

24. A method as recited in claim 17, the anti-viral and/or anti-bacterial composition protecting a practitioner from being infected with a communicable disease carried by the treated patient.

25. A method as recited in claim 17, the anti-viral and/or anti-bacterial composition protecting another patient from being infected with a communicable disease carried by the treated patient.
